(19) United States
(12) Patent Application Publication
(10) Pub. No.: US 2020/0268061 A1
(43) Pub. Date: Aug. 27, 2020

(54) TESTICLE GARMENT ASSEMBLY

(71) Applicant: Xavier Magdaleno, Englewood, CO (US)

(72) Inventor: Xavier Magdaleno, Englewood, CO (US)

(21) Appl. No.: 16/281,305

(22) Filed: Feb. 21, 2019

(51) Int. Cl. A41B 9/02

(52) U.S. Cl. CPC A41B 9/023; A61F 5/41

(57) ABSTRACT

A testicle garment assembly includes a bag that may be worn around a scrotum such that the bag forms a barrier between the scrotum and an adjacent upper thigh. It this way the bag inhibits the scrotum from engaging the adjacent upper thigh. The bag is comprised of a deformable material for minimizing compression of the scrotum. The bag is comprised of an air permeable material for inhibiting perspiration from forming on the scrotum. Additionally, the bag is comprised of a fluid absorbent material for wicking perspiration away from the scrotum. The bag has a volume of approximately 50.0 cubic cm to accommodate the average male scrotum. An elastic band is positioned on the bag and the elastic band biases the bag to close around the scrotum for retaining the bag around the scrotum.

### (b) CROSS-REFERENCE TO RELATED APPLICATIONS

### (c) STATEMENT REGARDING FEDERALLY SPONSORED RESEARCH OR DEVELOPMENT

Not Applicable

### (d) THE NAMES OF THE PARTIES TO A JOINT RESEARCH AGREEMENT

Not Applicable

### (e) INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### (f) STATEMENT REGARDING PRIOR DISCLOSURES BY THE INVENTOR OR JOINT INVENTOR

Not Applicable

### (g) BACKGROUND OF THE INVENTION

### (1) Field of the Invention

### (2) Description of Related Art Including Information Disclosed Under 37 CFR 1.97 and 1.98

The disclosure and prior art relates to garment devices and more particularly pertains to a new garment device for keeping a scrotum dry and comfortable.

### (h) BRIEF SUMMARY OF THE INVENTION

An embodiment of the disclosure meets the needs presented above by generally comprising a bag that may be worn around a scrotum such that the bag forms a barrier between the scrotum and an adjacent upper thigh. It this way the bag inhibits the scrotum from engaging the adjacent upper thigh. The bag is comprised of a deformable material for minimizing compression of the scrotum. The bag is comprised of an air permeable material for inhibiting perspiration from forming on the scrotum. Additionally, the bag is comprised of a fluid absorbent material for wicking perspiration away from the scrotum. The bag has a volume of approximately 50.0 cubic cm to accommodate the average male scrotum. An elastic band is positioned on the bag and the elastic band biases the bag to close around the scrotum for retaining the bag around the scrotum.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### (i) BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a top perspective view of a testicle garment assembly according to an embodiment of the disclosure.

FIG. 2 is a front view of an embodiment of the disclosure.

FIG. 3 is a back view of an embodiment of the disclosure.

FIG. 4 is a cross sectional view taken along line 4-4 of FIG. 1 of an embodiment of the disclosure.

FIG. 5 is a perspective in-use view of an embodiment of the disclosure.

### (j) DETAILED DESCRIPTION OF THE INVENTION

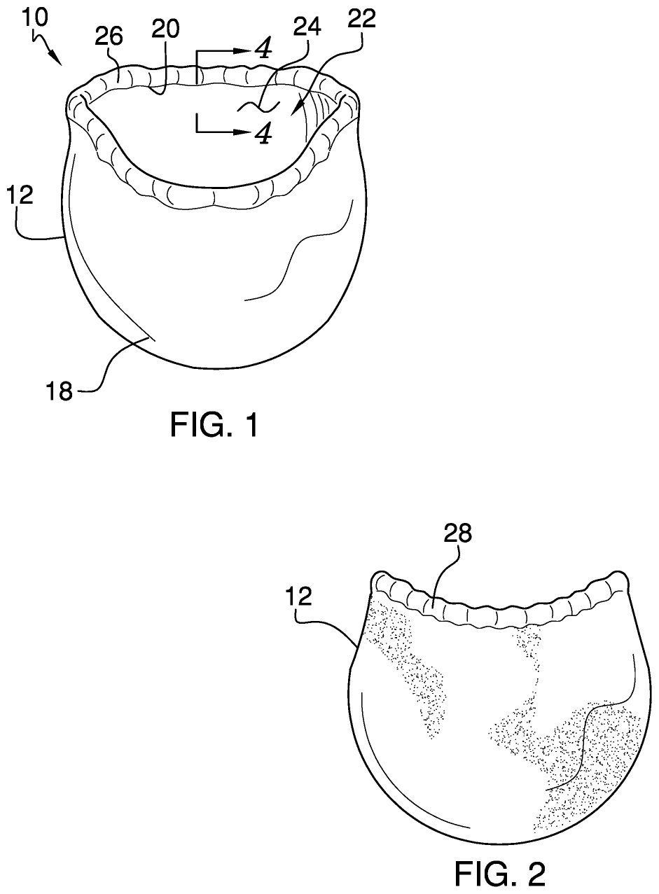

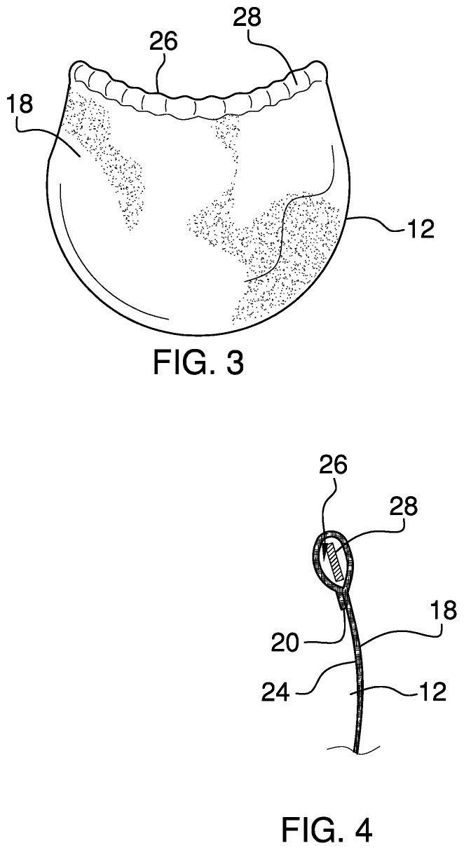

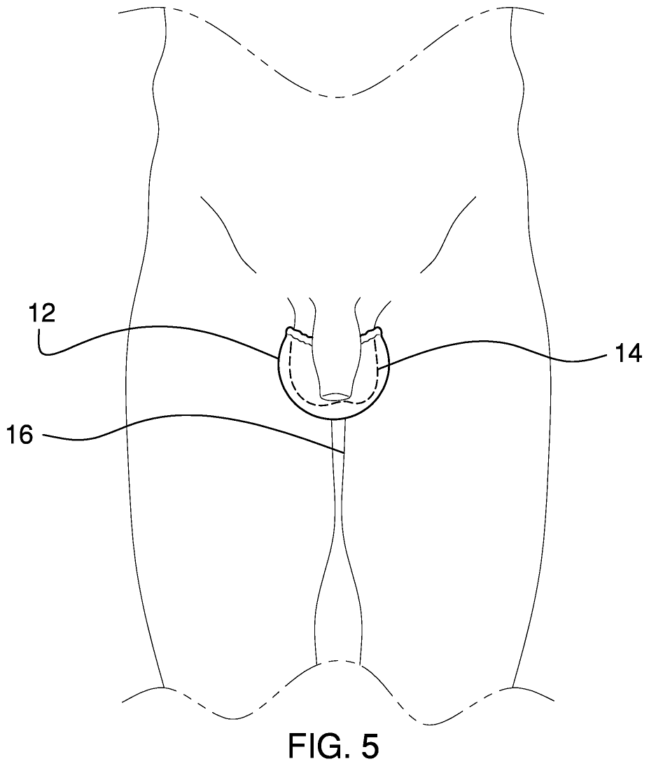

With reference now to the drawings, and in particular to FIGS. 1 through 5 thereof, a new garment device embodying the principles and concepts of an embodiment of the disclosure and generally designated by the reference numeral 10 will be described.

As best illustrated in FIGS. 1 through 5, the testicle garment assembly 10 generally comprises a bag 12 that is worn around a scrotum 14 to form a barrier between the scrotum 14 and an adjacent upper thigh 16. In this way the bag 12 inhibits the scrotum 14 from engaging the adjacent upper thigh 16. The bag 12 is comprised of a deformable material for minimizing compression of the scrotum 14. Additionally, the bag 12 is comprised of an air permeable material for inhibiting perspiration from forming on the scrotum 14. The bag 12 is comprised of a fluid absorbent material for wicking perspiration away from the scrotum 14. Moreover, the bag 12 has a volume of approximately 50.0 cubic cm for accommodating the average male scrotum 14.

The bag 12 has an outer wall 18 and a top edge 20, and the top edge 20 is continuously arcuate to form an opening 22 in the bag 12 to receive the scrotum 14. The outer wall 18 has an inside surface 24 and the outer wall 18 is folded over onto itself. The top edge 20 is attached to the inside surface 24 of the outer wall 18 to define a channel 26 extending around the opening 22. An elastic band 28 is positioned on the bag 12 and the elastic band 28 biases the bag 12 to close around the scrotum 14 for retaining the bag 12 around the scrotum 14. The elastic band 28 is positioned within the channel 26, the elastic band 28 is coextensive with the channel 26 and the elastic band 28 biases the opening 22 to a minimum diameter.

In use, the bag 12 is worn around the scrotum 14 prior to wearing underwear. In this way the bag 12 inhibits the scrotum 14 from contacting and thusly sticking to the adjacent upper thigh 16. Thus, the bag 12 enhances comfort for the user while driving or other activities involving sitting for extended periods of time. The bag 12 allows the scrotum 14 to breath and the bag 12 absorbs perspiration while the bag 12 is worn around the scrotum 14. Additionally, the bag 12 inhibits the scrotum 14 from soiling the underwear when the underwear is worn. The bag 12 is removed for bathing, sleeping and for any other reason.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A testicle garment assembly being configured to inhibit a user's scrotum from contacting the user's leg, said assembly comprising:
a bag being worn around a scrotum such that said bag forms a barrier between the scrotum and an adjacent upper thigh wherein said bag is configured to inhibit the scrotum from engaging the adjacent upper thigh, said bag being comprised of a deformable material for minimizing compression of the scrotum, said bag being comprised of an air permeable material for inhibiting perspiration from forming on the scrotum, said bag being comprised of a fluid absorbent material for wicking perspiration away from the scrotum, said bag having a volume of approximately 50.0 cubic cm wherein said bag is configured to accommodate the average male scrotum; and
an elastic band being positioned on said bag, said elastic band biasing said bag to close around the scrotum for retaining said bag around the scrotum.

2. The assembly according to claim 1, wherein said bag has an outer wall and a top edge, said top edge being continuously arcuate to form an opening in said bag wherein said opening is configured to receive the scrotum, said outer wall having an inside surface, said outer wall being folded over onto itself having said top edge being attached to said inside surface of said outer wall to define a channel extending around said opening.

3. The assembly according to claim 1, wherein:
said bag has an opening and a channel being coextensive with said opening; and
said elastic band is positioned within said channel, said elastic band being coextensive with said channel, said elastic band biasing said opening to a minimum diameter.

4. A testicle garment assembly being configured to inhibit a user's scrotum from contacting the user's leg, said assembly comprising:
a bag being worn around a scrotum such that said bag forms a barrier between the scrotum and an adjacent upper thigh wherein said bag is configured to inhibit the scrotum from engaging the adjacent upper thigh, said bag being comprised of a deformable material for minimizing compression of the scrotum, said bag being comprised of an air permeable material for inhibiting perspiration from forming on the scrotum, said bag being comprised of a fluid absorbent material for wicking perspiration away from the scrotum, said bag having a volume of approximately 50.0 cubic cm wherein said bag is configured to accommodate the average male scrotum, said bag having an outer wall and a top edge, said top edge being continuously arcuate to form an opening in said bag wherein said opening is configured to receive the scrotum, said outer wall having an inside surface, said outer wall being folded over onto itself having said top edge being attached to said inside surface of said outer wall to define a channel extending around said opening; and
an elastic band being positioned on said bag, said elastic band biasing said bag to close around the scrotum for retaining said bag around the scrotum, said elastic band being positioned within said channel, said elastic band being coextensive with said channel, said elastic band biasing said opening to a minimum diameter.
